(19) United States
(12) Patent Application Publication
(10) Pub. No.: US 2007/0148639 A1
(43) Pub. Date: Jun. 28, 2007

(54) Methods and devices for nucleic acid amplification on a surface

(76) Inventors: Alexander M. Chagovetz, Salt Lake City, UT (US); Steven M. Blair, Salt Lake City, UT (US)

Correspondence Address: TRASK BRITT, P.O. BOX 2550, SALT LAKE CITY, UT, 84110, US

(21) Appl. No.: 11/633,981

(22) Filed: Dec. 4, 2006

Related U.S. Application Data
(60) Provisional application No. 60/741,688, filed on Dec. 2, 2005.

(51) Int. Cl. C12Q 1/70; C12Q 1/68; C12P 19/34

(52) U.S. Cl. 435/5; 435/6; 435/91.2; 977/924

(57) ABSTRACT

Described are methods and devices for immobilizing primers on the surface of a microarray with polymer linkers. Nucleic acid amplification and total analysis without the necessity of polymerase chain reaction may be accomplished with the disclosed methods and devices.

### CROSS-REFERENCE TO RELATED APPLICATION

This application claims the benefit of the filing date of U.S. Provisional Patent Application 60/741,688 filed on Dec. 2, 2005, the contents of the entirety of which is incorporated by this reference.

### TECHNICAL FIELD

The invention generally relates to biotechnology, and, more specifically, to the field of diagnostics, such as nucleic acid amplification on a surface.

### BACKGROUND

Nucleic acid amplification in solution-based reactions, either through thermal cycling (e.g., polymerase chain reaction “PCR” and its modifications) or isothermal amplification (e.g., rolling circle amplification “RCA,” Ionian method, Invader) is well established and has been widely used for the last 25 years. There is, however, an intrinsic limitation in solution-based reactions with respect to multiplexing, due to multiple competitive processes, which introduce bias in quantitative features (concentrations) of multiple targets. Two approaches emerged to overcome these limitations: non-specific whole genome amplification through the use of short scrambled primers or amplifications based on generic oligoT primer (and its permutations) in conjunction with scrambled primers for messenger ribonucleic acid (“mRNA”) amplifications. In all cases, reaction products are interrogated through post-amplification techniques: arrayed capture probes, electrophoresis, or solution-based deoxyribonucleic acid (“DNA”) specific dyes (e.g., minor groove binders, major grove binders, intercalators).

Recent advances in attempts to overcome competitive interactions of solution based amplification include separating amplifications in half reactions between solution reactions and interface-based (immobilized) reactions, where half of the primers are in solution, while the other half are immobilized in/on the interface (hydrogels, membranes of organic (nitrocellulose) or inorganic (Al₂0₃) origin). Although advantageous from point of view of separating reactions, there methods are marginally productive, since competition and different efficiencies of amplification still contribute to resulting quantitative biases.

U.S. Pat. No. 5,641,658, filed Aug. 3, 1994, the contents of the entirety of which are incorporated by this reference, discloses a method for performing amplification of a nucleic acid with two primers bound to a single solid support.

### SUMMARY OF THE INVENTION

Embodiments of the invention include methods and devices for performing surface-based amplification with greater speed and greater fidelity than solution-based methods such as PCR. The invention overcomes certain limitations of competitive processes in solution-based amplification methods. The invention may be used with chip-based microarrays and total analysis without the necessity of PCR.

Certain embodiments of the invention involve an interface modified with linkers of appropriate length and rotational mobility. The linkers are functionalized in order to achieve immobilization on the interface on one end, and have a different feature on the other end for the attachment of primers. A pair of primers (first and second primers), corresponding to each individual nucleic acid target strand, are co-immobilized within the confines of one reaction zone. The appropriate first primers in each reaction zone capture target strands. Enzymatic reaction extends the primers providing a complementary copy of each target strand. The original target strands are separated from the primers and the extension products extending from the primers. The length of the linkers is chosen in such a way to allow interactions between the second primers and the single-stranded extension product of the first primers. The immobilized extension products are cross-primed with the second primers. Enzymatic reaction extends the second primers providing a copy of each target strand. Repetition of this embodiment may amplify the target strand within in each reaction zone.

In certain embodiments, the invention includes a method comprising: creating a reaction zone of a solution interface with a plurality of linkers; immobilizing primer pairs for complementary nucleic acid targets to the plurality of linkers; flooding the reaction zone with a variety of nucleic acids containing the complementary nucleic acid targets; capturing the complementary nucleic acid targets with the immobilized primer pairs; extending the immobilized primers; separating off each of the complementary nucleic acid targets; and cross-priming the extended immobilized primers with some remaining unextended immobilized primer pairs.

In certain embodiments, the invention includes a method of amplifying a nucleic acid target strand, the method comprising: modifying an interface with at least two polymer linkers; immobilizing a primer pair corresponding to a nucleic acid target strand to the at least two polymer linkers; flooding the reaction zone with a variety of nucleic acids including the nucleic acid target strand; capturing the nucleic acid target with a first primer of the immobilized primer pair; extending the first primer; separating off the nucleic acid target strand; and cross-priming the extended first primer with a second primer of the immobilized primer pair.

### BRIEF DESCRIPTION OF THE SEVERAL VIEWS OF THE DRAWINGS

FIG. 1a illustrates primers and linker attached to an interface with target strands in solution.

FIG. 1b illustrates hybridization of target strand to primers.

FIGS. 2a and 2b illustrate extension of primers.

FIG. 3 illustrates removal of target strands from the extension product of primers.

FIGS. 4a and 4b illustrate hybridization of a primer extension product.

FIG. 5 illustrates immobilized double-stranded products on an interface.

### DETAILED DESCRIPTION OF THE INVENTION

Embodiments of the invention include methods and devices for performing surface-based amplification with better speed and greater fidelity than solution-based methods such as PCR. The invention overcomes limitations of competitive processes in solution-based amplification methods. The invention may be used with chip-based microarrays and total analysis without the necessity of PCR.

Certain embodiments of the invention appear to overcome inherent limitations of solution-based amplifications through confining reactions to the interfaces. One example of an interface includes the surface of a microarray. However, other surfaces may form the interface as well. Sensitivity of detection may be elevated by choosing enhanced interfaces. Possible enhanced interfaces include: membranes. thin film planar waveguides, fiber optics guides, surface modifications with polymeric or inorganic porous beads, nanoparticles and nanocavities, and efficient selective excitation substrates (e.g., evanescent field).

Certain embodiments of the invention involve limiting the mobility of one of several groups of analytes in a solution. Possible analytes that may be immobilized include primers and extension products of the primers. Movement of other groups of analytes in the solution is not restricted. Possible unrestricted analytes include enzymes, salts, and nucleotide triphosphates.

Reference will now be made to the drawings wherein like numerals represent like elements. The drawings are not necessarily to scale.

As illustrated in FIG. 1a, an interface 10 may be modified with linkers 20 of appropriate length and rotational mobility. Examples of linkers 20 include organic linear polymers and dendrimers. Linkers 20 may be functionalized in order to achieve immobilization on the interface. Immobilization may be accomplished via chemical or affinity interactions. Linkers 20 may have features on the non-immobilized end for attachment to primers 30. Attachment between the linkers 20 and primers 30 may be accomplished by chemical or affinity attachment or by any other method known in the art.

A pair of primers (first primer 32 and second primer 34), corresponding to each individual nucleic acid target strand, are co-immobilized within the confines of a reaction zone 100. The first primer 32 and second primer 34 may be identical. Alternatively, the first primer 32 may be a forward primer and the second primer 34 may be a reverse primer, or vice versa. Each reaction zone 100 may be the spot of a microarray. Each reaction zone 100 may also be addressable. The reaction zones 100 may be formed on the interface 10 such that no primer-primer interactions are possible outside each reaction zone 100 (e.g., between reaction zones). The primers 30 may be immobilized at equimolar quantities, but this is not mandatory.

The length of linkers 20 may be chosen in such a way that the linkers 20 allow interactions between the primers 30 and single-stranded extension products through limited mobility, such as limited gyration, of the appropriate linkers 20.

The nucleic acid target strands 42 and 44 may be DNA (genomic or non-genomic), mRNA, ribosomal RNA, viral RNA, non-naturally occurring nucleic acids, or nucleic acid samples of other origin and structure.

As illustrated in FIGS. 1a and 1b, first target strand 42 and second target strand 44 may be captured by first primer 32 and second primer 34, respectively. Capturing may be accomplished by hybridization. The first target strand 42 may be identical to the second target strand 44. Alternatively, the first target strand 42 may be complementary to the second target strand 44. Additionally, in another embodiment, only the first target strand 42 may be present.

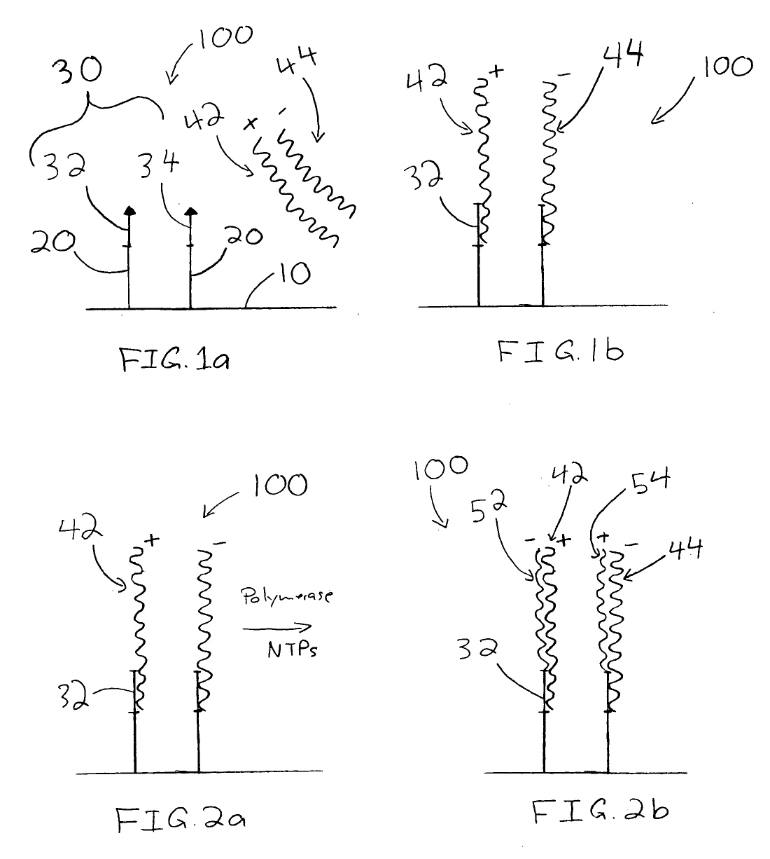

As illustrated in FIGS. 2a and 2b, the first primer 32 and second primer 34 may be extended through enzymatic reaction (e.g., polymerization) to form first extension product 52 and second extension product 54. The enzymatic reaction may be accomplished with polymerase, including transcriptases, and nucleotide triphosphates (“NTPs”). The first extension product 52 is complementary to the first target strand 42. The second extension product 54 is complementary to the second target strand 44. The first extension product 52 and second extension product 54 may be identical or complementary. Any method of extending a primer may be used.

As illustrated in FIG. 3, first target strand 42 and second target strand 44 are separated from the first extension product 52 and second extension product 54 respectively. The first extension product 52 and second extension product 54 (at least at this stage) remains attached to the primers 30. The separation of the target strands 40 from the extension products 50 may be accomplished by heating, such as by following the thermal cycling protocol. Separation may also be accomplished by enzymatic action (e.g., endonuclease or helicase activity). Any method of separating an extension product from a target strand may be used.

The second extension product 54 may be complementary to the first extension product 52. The second extension product 54 may be identical to the first target strand 42. The first extension product 52 may be identical to the second target strand 44.

The separated target strands 40 may remain in the reaction zone 100 for further reaction with other immobilized primers 30. Multiple repetitions of hybridizing the target strands 40 with primers 30 and then extending those primers 30 may result in exponential target strand 40 amplification within the confines of the reaction zone 100.

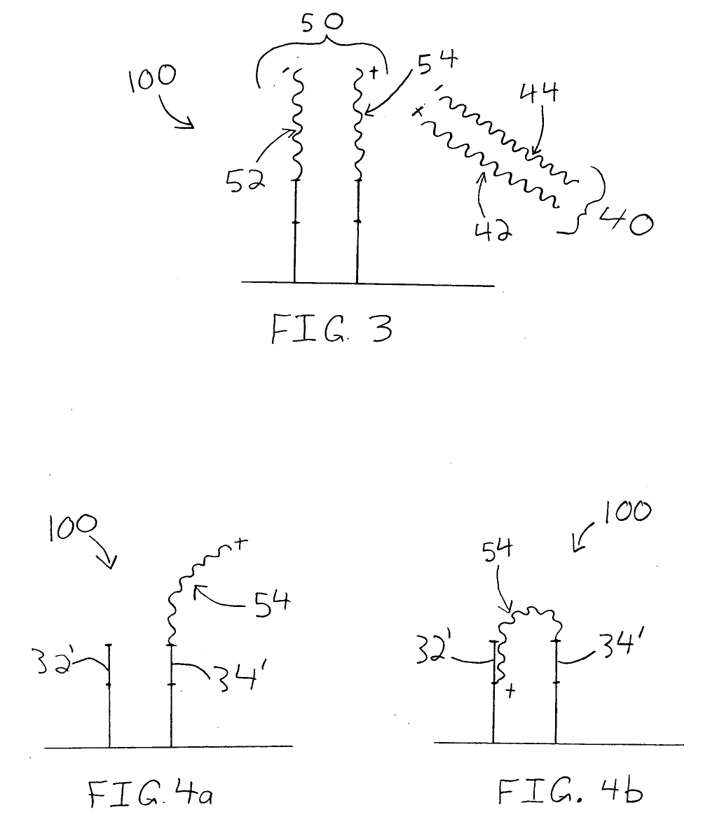

As illustrated in FIGS. 4a and 4b, an immobilized second extension product 54 may be cross-primed with a first primer 32′. The first primer 32′ may be a primer to which a target strand 42 had not previously hybridized, but an adjoining second primer 34 had hybridized with a target strand 44 and enzymatically extended. Alternatively, the first primer 32′ may be a primer on which a first extension product 52 was formed, but the first extension product has been cleaved, such as by a restriction endonuclease.

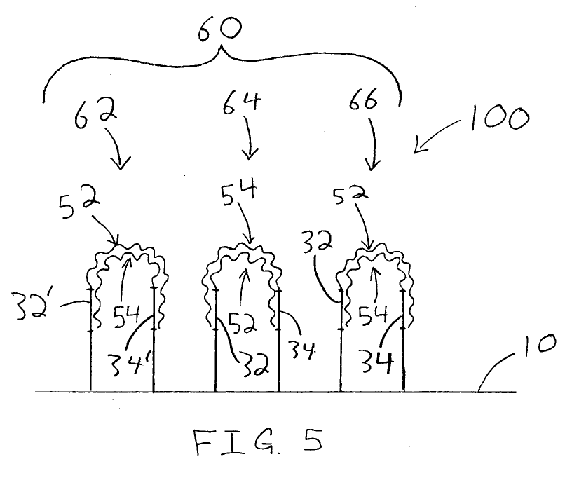

Referring to FIG. 5, the first primer 32′ may be extended through enzymatic reaction forming a first extension product 52. First extension product 52 and second extension product 54 may hybridize to form immobilized double-stranded product 62. Immobilized double-stranded product 64 and 66 may be formed by the hybridization of the extension products 50 of FIG. 3.

Immobilized double-stranded product 62, 64, and 66 may be identical. For example, the embodiment illustrated in FIG. 3 may be completed at the same time that the embodiment illustrated in FIG. 4a is formed. Reaction zone 100 may then be placed in hybridizing conditions (e.g., temperature decreased). The embodiment illustrated in FIG. 3 may hybridize to form immobilized double-stranded product 64. Meanwhile, the embodiment illustrated in FIG. 4a may hybridize with first primer 32′ to form the embodiment illustrated in FIG. 4b. A further enzymatic reaction will result in the embodiment illustrated in FIG. 4b forming immobilized double-stranded product 62.

The above actions may be repeated multiple times forming a plurality of immobilized double-stranded products 60 on interface 10 within reaction zone 100.

The above actions may be coupled with fluidic delivery of solution-based reagents for enhanced mass-transport and real-time methods of nucleic acid detection. Examples of real-time detection that may be used include the use of: fluorescent dyes, fluorescent resonance energy transfer (“FRET”), fluorescence quenching, fluorescence polarization, fluorescence lifetime, and chemiluminescence (e.g., sandwich methods). Additionally, end-point analysis may also be conducted.

While disclosed with particularity, the foregoing methods and devices are more fully explained and the invention described by the following claims. It is clear to one of ordinary skill in the art that numerous and varied alterations can be made to the foregoing methods and devices without departing from the spirit and scope of the invention. Therefore, the invention is only limited by the claims.

What is claimed is:

1. A method comprising:
creating a reaction zone of a solution interface with a plurality of linkers;
immobilizing primer pairs for complementary nucleic acid targets to the plurality of linkers;
flooding the reaction zone with a variety of nucleic acids containing the complementary nucleic acid targets;
capturing the complementary nucleic acid targets with the immobilized primer pairs;
extending the immobilized primers;
separating off each of the complementary nucleic acid targets; and
cross-priming the extended immobilized primers with some remaining unextended immobilized primer pairs.

2. The method according to claim 1, further comprising:
extending the cross-primed extended immobilized primers.

3. The method according to claim 2, further comprising performing repeatedly:
extending the immobilized primers;
separating off the specific nucleic acid targets; and
cross-priming the extended immobilized primers with some remaining unextended immobilized primer pairs.

4. The method according to claim 1, wherein flooding the zone with the variety of nucleic acids containing the specific nucleic acid targets comprises flooding the zone with genomic DNA, mRNA, ribosomal RNA, viral RNA or nucleic acid samples of other origin and structure.

5. The method according to claim 1, further comprising attaching the linkers to the interface via chemical or affinity attachment.

6. The method according to claim 1, where modifying the reaction zone of the solution interface with the plurality of linkers comprises modifying a reaction zone of a solution interface with linear polymers or dendrimers.

7. The method according to claim 1, further comprising denaturing double-stranded nucleic acids to form complementary nucleic acid targets.

8. A method of amplifying a nucleic acid target strand, the method comprising:
modifying an interface with at least two polymer linkers;
immobilizing a primer pair corresponding to a nucleic acid target strand to the at least two polymer linkers;
flooding the reaction zone with a variety of nucleic acids including the nucleic acid target strand;
capturing the nucleic acid target with a first primer of the immobilized primer pair;
extending the first primer;
separating off the nucleic acid target strand; and
cross-priming the extended first primer with a second primer of the immobilized primer pair.

9. The method according to claim 8, further comprising:
extending the cross-primed second primer.

10. The method according to claim 9, where extending the cross-primed second primer comprises forming a nucleic acid identical to the nucleic acid target strand.

11. The method according to claim 8, further comprising performing repeatedly:
extending the first primer;
separating off the nucleic acid target strand; and
cross-priming the extended first primer with the second primer of the immobilized primer pair.

12. The method according to claim 8, wherein flooding the zone with a variety of nucleic acids containing the specific nucleic acid targets comprises flooding the zone with deoxyribonucleic acids (“DNA”), ribonucleic acids (“RNA”), or non-naturally occurring nucleic acids.

13. The method according to claim 8, further comprising attaching the linkers to the interface via affinity attachment.

14. The method according to claim 8, wherein modifying the interface with at least two polymer linkers comprises modifying the interface with at least two linear polymer linkers or dendrimer linkers.

15. A microarray comprising:
a substrate surface comprising a plurality of spots, each spot including a plurality of polymer linkers; and
a plurality of primers attached to the plurality of polymer linkers;

16. The microarray of claim 15 further comprising, nucleic acid target strands hybridized to the plurality of primers.

17. The microarray of claim 15, wherein the plurality of primers attached to the plurality of polymer linkers comprises a forward primer attached to one of the plurality of polymer linkers and a reverse primer attached to an adjoining one of the plurality of polymer linkers.

18. The microarray of claim 15, wherein each of the plurality of primers for a single spot are identical.

19. The microarray of claim 15, where the substrate comprises membranes, thin film planar waveguides, fiber optics guides, surface modifications with polymeric or inorganic porous beads, nanoparticles and nanocavities, or efficient selective excitation substrates.

20. The microarray of claim 15, further comprising detection equipment for detecting nucleic acid targets via fluorescent dyes, FRET, fluorescence quenching, fluorescence polarization, fluorescence lifetime, and/or sandwich methods.
